(19) United States
(12) Patent Application Publication
(10) Pub. No.: US 2014/0039463 A1
(43) Pub. Date: Feb. 6, 2014

(54) Adhesion Enhancement Additives for Liquid Crystal Polymer Compositions and Methods Relating Thereto

(71) Applicant: Ticona LLC, Florence, KY (US)

(72) Inventors: Young Shin Kim, Erlanger, KY (US); Bernard Jason Smith, Cincinnati, OH (US); Sung Hye Kim, Erlanger, KY (US)

(73) Assignee: Ticona LLC, Florence, KY (US)

(21) Appl. No.: 13/955,565

(22) Filed: Jul. 31, 2013

Related U.S. Application Data
(60) Provisional application No. 61/678,361, filed on Aug. 1, 2012.

(51) Int. Cl. C09K 19/54; A61M 25/00; H01L 31/0203

(52) U.S. Cl. CPC C09K 19/542; H01L 31/0203; A61M 25/0043
USPC 604/523; 428/343; 428/36.91; 428/394; 428/355 AC; 428/337; 428/354; 428/40.1; 428/520; 428/522; 428/35.2; 428/172; 136/259; 383/116; 427/162; 156/332

(57) ABSTRACT

A liquid crystal polymer composition may comprise a liquid crystal polymer and an adhesion enhancement compound. Such a liquid crystal polymer may be utilized in layered articles, molded articles, and like and products like solar cells, optoelectronic structures, medical equipment, and tubing. Further, ethylene vinyl acetate adhesives may be utilized in some of the layered articles.

### BACKGROUND

The present invention relates to liquid crystal polymer compositions that comprise adhesion enhancement compounds, and methods, articles, and products relating thereto.

Liquid crystal polymers (“LCPs”) are unique among polymers because they typically have very high tensile, flexural, and temperature resistance properties, which may be desirable properties for high performance applications, such as, structural applications and electronic applications. However, LCP materials have a nonpolar surface with a low surface energy, which limits the ability for adhesives to adhere to LCP materials, and consequently adhere LCP materials to other materials. Several approaches have been utilized to implement LCP materials in applications that require adhesion to another material, two of which being (1) strong adhesives and (2) surface functionalization of the LCP materials.

Most often, adhesives used in conjunction with LCP materials have been urethane-based adhesives. However, some of the drawbacks of urethane-based adhesives, which have limited some applications of LCP materials, include the use of formaldehyde solvents during production and limited flexible movement in the final product due to the stiffness of the hardened or cured adhesive. Formaldehyde solvents sometime release over time as the LCP materials are used, which is undesirable because formaldehyde is a known carcinogen.

A second approach to improve adhesion involves introducing surface functionality to the LCP material through plasma irradiation, excimer laser irradiation, UV irradiation, and corona treatment. However, not only are the surface treatments commercially impracticable, but the surface treatments have also not been successful in yielding greatly improved LCP adhesion properties.

Accordingly, LCP materials that enable the use of a variety of different adhesives may enable expanded applications of LCP materials in addition to reducing the use of and potential exposure to formaldehyde-based solvents.

### SUMMARY OF THE PRESENT INVENTION

The present invention relates to liquid crystal polymer compositions that comprise adhesion enhancement compounds, and methods, articles, and products relating thereto.

In one embodiment of the present invention, an article may comprise: a liquid crystal polymer article component that comprises a liquid crystal polymer composition that comprises a liquid crystal polymer and an adhesion enhancement compound; and an adhesive layer disposed on at least a portion of the liquid crystal polymer article component.

In another embodiment of the present invention, a layered article may comprise: a liquid crystal polymer layer comprising a liquid crystal polymer and an adhesion enhancement compound; an adhesive layer comprising a first ethylene vinyl acetate copolymer; and an encapsulant layer comprising a second ethylene vinyl acetate copolymer.

In another embodiment of the present invention, a layered article may comprise: a first liquid crystal polymer layer comprising a first liquid crystal polymer and a first adhesion enhancement compound; a first adhesive layer comprising ethylene vinyl acetate copolymer; a second liquid crystal polymer layer comprising a second liquid crystal polymer and a second adhesion enhancement compound; a second adhesive layer comprising ethylene vinyl acetate copolymer; a third liquid crystal polymer layer comprising a third liquid crystal polymer and a third adhesion enhancement compound; and a third adhesive layer comprising ethylene vinyl acetate copolymer.

In yet another embodiment of the present invention, a method may comprise: applying an adhesive comprising to a liquid crystal polymer layer that comprises a liquid crystal polymer and an adhesion enhancement compound.

In yet another embodiment of the present invention, a method may comprise: extruding an adhesive layer comprising ethylene vinyl acetate copolymer onto a liquid crystal polymer layer comprising a liquid crystal polymer and an adhesion enhancement compound.

In another embodiment of the present invention, a method may comprise: laminating an adhesive layer between a liquid crystal polymer layer and a second layer, the adhesive layer comprising ethylene vinyl acetate copolymer, and the liquid crystal polymer layer comprising a liquid crystal polymer and an adhesion enhancement compound.

In yet another embodiment of the present invention, a method may comprise: arranging a plurality of layers such that at least two layers are an adhesive layer adjacent to a liquid crystal polymer layer, the adhesive layer comprising ethylene vinyl acetate copolymer, and the liquid crystal polymer layer comprising a liquid crystal polymer and an adhesion enhancement compound; and laminating the plurality of layers together to form a layered article.

In one embodiment of the present invention, a catheter may comprise: a liquid crystal polymer layer comprising a liquid crystal polymer and an adhesion enhancement compound; an adhesive layer disposed about at least a portion of the liquid crystal polymer layer, the adhesive layer comprising ethylene vinyl acetate copolymer; and a thermoplastic layer disposed about the adhesive layer.

In another embodiment of the present invention, an optoelectronic structure may comprise, in order: a liquid crystal polymer layer comprising a liquid crystal polymer and an adhesion enhancement compound; an adhesive layer disposed on at least a portion of the liquid crystal polymer layer, the adhesive layer comprising ethylene vinyl acetate copolymer; and an optoelectronic device.

In yet another embodiment of the present invention, a blood bag may comprise, in order: a first thermoplastic polymer layer; a first adhesive layer; a liquid crystal polymer layer comprising a liquid crystal polymer and an adhesion enhancement compound; a second adhesive layer disposed on at least a portion of the liquid crystal polymer layer; and a first thermoplastic polymer layer.

In another embodiment of the present invention, a piece of surgical equipment may comprise: a tube comprising a liquid crystal polymer layer comprising a liquid crystal polymer and an adhesion enhancement compound.

In yet another embodiment of the present invention, a channeled structure may comprise at least one channel formed by a molded liquid crystal polymer composition adhered to a substrate with an adhesive, the molded liquid crystal polymer composition comprising liquid crystal polymer and an adhesion enhancement compound.

The features and advantages of the present invention will be readily apparent to those skilled in the art upon a reading of the description of the preferred embodiments that follows.

### DESCRIPTION OF DRAWINGS

FIG. 1A illustrates a cross-section of a medical tubing according to at least one embodiment of the present invention.

FIG. 1B illustrates a medical tubing according to at least one embodiment of the present invention being used in conjunction with a peristaltic pump.

FIG. 2 illustrates a cross-section of a portion of an optoelectronic structure according to at least one embodiment of the present invention.

FIG. 3A illustrates a cross-section of a portion of a bio-container according to at least one embodiment of the present invention.

FIG. 3B illustrates a bio-container according to at least one embodiment of the present invention.

FIG. 3C illustrates a cross-section of a portion of a bio-container according to at least one embodiment of the present invention.

FIG. 4 illustrates a cross-section of a portion of a solar cell according to at least one embodiment of the present invention.

FIG. 5A illustrates a cross-section of an electrical wire according to at least one embodiment of the present invention.

FIG. 5B illustrates a cross-section of an electrical wire according to at least one embodiment of the present invention.

### DETAILED DESCRIPTION

The present invention relates to liquid crystal polymer compositions that comprise adhesion enhancement compounds, and methods, articles, and products relating thereto.

The present invention provides for, in some embodiments, liquid crystal polymer compositions (“LCP compositions”) that more readily adhere to adhesives, including non-urethane-based adhesives like ethylene vinyl acetate copolymer adhesives. The use of ethylene vinyl acetate copolymer adhesives in conjunction with LCP compositions of the present invention may, in some embodiments, advantageously enable articles and products with structural flexibility while utilizing a more environmentally-friendly adhesive, as compared to the formaldehyde solvents utilized in urethane-based adhesives. Further, ethylene vinyl acetate copolymer may be produced in a pharmaceutical grade, thereby enabling a plurality of medical applications for the LCP compositions of the present invention.

Further, LCP compositions of the present invention may also readily adhere to solder, which may expand the electronics applications currently available to liquid crystal polymers.

It should be noted that when “about” is provided herein at the beginning of a numerical list, “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit.

### I. Liquid Crystal Polymer Compositions

In some embodiments, LCP compositions of the present invention may comprise liquid crystal polymers (“LCPs”) and adhesion enhancement compounds.

In some embodiments, suitable LCPs for use in conjunction with LCP compositions of the present invention may comprise monomeric units that include, but are not limited to, Formula I, Formula II, Formula III, Formula IV, Formula V, Formula VI, Formula VII, and the like, and any combination thereof.

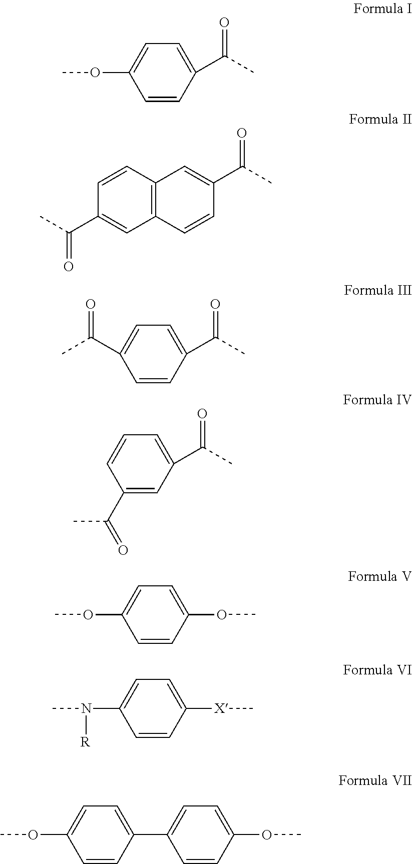

wherein X′ may be NR′, CO, O, and any combination thereof; and wherein R and R′ may independently be H, alkyl groups having 1 to 4 carbon atoms, fluoroalkyl groups having 1 to 4 carbon atoms, phenyl, and any combination thereof.

In some embodiments, LCPs for use in conjunction with LCP compositions of the present invention may have an intrinsic viscosity ranging from a lower limit of about 0.1 dl/g, 0.5 dl/g, 1 dl/g, 2 dl/g, or 4 dl/g to an upper limit of about 10 dl/g, 8 dl/g, or 5 dl/g, and wherein the intrinsic viscosity may range from any lower limit to any upper limit and encompass any subset therebetween.

In some embodiments, adhesion enhancement compounds may by molecules having a molecular weight less than about 1,000 amu and a decomposition temperature above 350° C. As used herein, the term “heteroatom moiety” refers to a functional group that comprises at least one heteroatom selected from the group consisting of O, N, S, P, and any combination thereof. Examples of suitable heteroatom moieties may include, but are not limited to, carboxyl groups, hydroxyl groups, aldehyde groups, ester groups, amino groups, amido groups, carboxyamido groups, imido groups, thiol groups, sulfinyl groups, sulfino groups, and the like.

In some embodiments, suitable adhesion enhancement compounds for use in conjunction with LCP compositions of the present invention may include, but are not limited to, 2,6-naphthalene dicarboxylic acid, 6-hydroxyl naphthalene 2-carboxylic acid, p-hydroxybenzoic acid, terephthalic acid, isophthalic acid, benzenediol, a bisphenol, 4-aminophenol, acetaminophen, styrene, α-methyl styrene, halogenated styrene, acrylonitrile, methylacrylonitrile, acrylamide, methacrylamide, vinyl naphthalene, N-methylol acrylamide, vinyl acetate, vinylchloride, vinylidene chloride, benzyl acrylate, methacrylic acid, itaconic acid, fumaric acid, maleic acid, the small molecules listed in Formulas VIII-XV below, and the like, any derivative thereof, and any combination thereof.

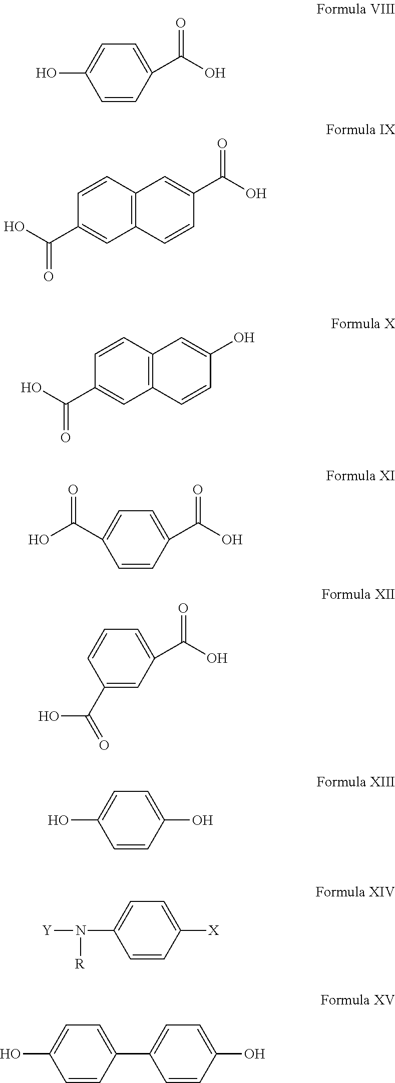

wherein X may be NY′R′, COOH, OH, and any combination thereof; wherein Y and Y′ may independently be H, HOCH₃, and any combination thereof, and wherein R and R′ may independently be H, alkyl groups having 1 to 4 carbon atoms, fluoroalkyl groups having 1 to 4 carbon atoms, phenyl, and any combination thereof.

In some embodiments, the concentration of the adhesion enhancement compounds in LCP compositions of the present invention may range from a lower limit of about 0.01%, 0.05%, 0.1%, 0.5%, or 1% to an upper limit of about 30%, 20%, 10%, 8%, or 5% by weight of the LCP, and wherein the concentration of the adhesion enhancement compounds may range from any lower limit to any upper limit and encompass any subset therebetween.

In some embodiments, LCP compositions may comprise LCPs, adhesion enhancement compounds, and additives. Suitable additives for use in conjunction with LCP compositions of the present invention may include, but are not limited to, reinforcing agents, thermoplastic resins, mold release-improving agents (e.g., fluorine resins, metal soaps, and the like), nucleating agents, antioxidants, stabilizers, plasticizers, slipping agents, color protection agents, coloring agents, ultraviolet light absorbers, antistatic agents, lubricants, flame retardants, and the like, and any combination thereof.

In some embodiments, suitable reinforcing agents for use in conjunction with LCP compositions of the present invention may include, but are not limited to, glass fibers (e.g., milled glass fiber, and chopped glass fiber), inorganic fillers (e.g., glass beads, hollow glass bulbs, glass powder, mica, talc, clay, silica, alumina, potassium titanate, wollastonite, calcium carbonate (heavy, light, and colloidal), magnesium carbonate, basic magnesium carbonate, sodium sulfate, calcium sulfate, barium sulfate, calcium sulfite, aluminum hydroxide, magnesium hydroxide, calcium hydroxide, calcium silicate, silica sand, silica rock, quartz, titanium oxide, zinc oxide, iron oxide graphite, molybdenum, asbestos, silica alumina fiber, alumina fiber, gypsum fiber, carbon fiber, carbon black, white carbon, diatomaceous earth, bentonite, sericite, white sand, and black lead), metal or nonmetal whisker (e.g., potassium titanate whisker, alumina whisker, aluminum borate whisker, silicon carbide whisker, and silicon nitride whisker), and the like, and any combination thereof. In some embodiments, reinforcing agents may preferably include glass fiber, glass powder, mica, talc, carbon fiber, and the like.

In some embodiments, reinforcing agents may have a surface treatment. In some embodiments, reinforcing agents may have surface treatment agents adsorbed to the reinforcing agent surface. Examples of surface treatment agents for use in conjunction with reinforcing agents may include, but are not limited to, reactive coupling agents (e.g., silane coupling agents, titanate coupling agents, and borane coupling agents), lubricants (e.g., higher fatty acids, higher fatty acid esters, metal salts of higher fatty acid, and fluorocarbon surfactants), and the like, and any combination thereof.

In some embodiments, reinforcing agents may be included in LCP compositions of the present invention in an amount ranging from a lower limit of about 1%, 5%, 10%, or 25% by weight of LCP to an upper limit of about 400%, 200%, 100%, or 50% by weight of LCP, and wherein the amount of reinforcing agent may range from any lower limit to any upper limit and encompass any subset therebetween.

In some embodiments, suitable thermoplastic resins for use in conjunction with LCP compositions of the present invention may include, but are not limited to, polycarbonates, polyamides, polysulfones, polyphenylene sulfides, polyphenylene ethers, polyether ketones, polyetherimides, polycyclohexylene-dimethylene terephthalate, and the like, any derivative thereof, and any combination thereof.

In some embodiments, an LCP composition having a water vapor transmission rate of about 3 g/m²/day or less, as measured by ASTM R1249-06 with a 25 micron thick sample of the LCP composition. In some embodiments, an LCP layer may comprise an LCP composition having a water vapor transmission rate ranging from a lower limit of about 0.001 g/m²/day, 0.05 g/m²/day, or 0.1 g/m²/day to an upper limit of about 3 g/m²/day, 1 g/m²/day, or 0.5 g/m²/day, as measured by ASTM R1249-06 with a 25 micron thick sample of the LCP composition, and wherein the water vapor transmission rate may range from any lower limit to any upper limit and encompass any subset therebetween.

In some embodiments, an LCP composition having an oxygen permeability of about 3 g/m²/day or less, as measured by ASTM D3985 with a 25 micron thick sample of the LCP composition. In some embodiments, an LCP layer may comprise an LCP composition having an oxygen permeability ranging from a lower limit of about 0.001 g/m²/day, 0.05 g/m²/day, or 0.1 g/m²/day to an upper limit of about 3 g/m²/day, 1 g/m²/day, or 0.5 g/m²/day, as measured by ASTM D3985 with a 25 micron thick sample of the LCP composition, and wherein the oxygen permeability may range from any lower limit to any upper limit and encompass any subset therebetween.

In some embodiments, LCP compositions of the present invention may comprise liquid crystal polymers described herein, adhesion enhancement compounds described herein, and optionally at least one of the additives described herein (including any combination of additives), each in a desired amount including the amounts described herein. Further, in some embodiments, LCP compositions of the present invention may have a desired water vapor transmission rate and/or a desired oxygen permeability.

### II. Articles Comprising LCP Compositions and Methods Relating Thereto

In some embodiments, articles of the present invention may comprise LCP compositions according to any embodiments described herein. Suitable articles may include, but are not limited to, molded articles, layered articles, and the like, and any combination thereof. It should also be noted that the terms “layers,” “films,” “coatings,” and the like do not necessarily indicate an absolute thickness or relative thickness. For example, layers may have holes, e.g., webs, nets, lattices, grids, discontinuous layers (e.g., lightly sprayed on adhesives), and the like. Further, the terms “layers,” “films,” “coatings,” and the like do not necessarily indicate a specific shape, that is, these terms are not limited to flat sheets.

Some embodiments may involve forming LCP articles that comprising LCP compositions of the present invention by combining the constituents of the desired LCP compositions (e.g., LCPs, adhesion enhancement compounds, and optionally additives) to yield an LCP composition precursor and forming the LCP composition precursor into a desired form (e.g., a molded article, a tube, a fiber, a billet, a sheet, and any hybrid thereof) thereby yielding an LCP article or component thereof. In some embodiments, forming LCP articles of the present invention may further include disposing an additional layer on the LCP article component.

In some embodiments, combining the constituents of the desired LCP composition may involve heating, mixing, kneading, and any combination thereof. Suitable apparatuses for combining the constituents of the desired LCP composition may include, but are not limited to, a single screw extruder, a twin-screw extruder, a Banbury mixer, a roller, a Brabender, a kneader, and the like.

In some embodiments, forming the LCP composition precursor into an LCP article or component thereof may involve at least one of compression molding, compression molding then skiving, injection molding, blow molding, casting, extruding, and the like, and any hybrid thereof.

In some embodiments, an LCP article or component thereof may be in the form of a sheet and referred to as “an LCP layer.” In some embodiments, an LCP layer described herein may have a thickness ranging from a lower limit of about 1 micron, 5 microns, 10 microns, 25 microns, or 50 microns to an upper limit of about 1500 microns, 1000 microns, 500 microns, or 100 microns, and wherein the thickness may range from any lower limit to any upper limit and encompass any subset therebetween.

In some embodiments, LCP articles of the present invention may comprise an LCP article component (e.g., a molded article, a tube, a fiber, a billet, and a sheet) and an additional layer disposed on at least a portion of the LCP article component. In some embodiments, LCP articles of the present invention may comprise an LCP layer and an additional layer disposed on at least a portion of the LCP article component.

In some embodiments, suitable additional layers for use in conjunction with layered articles comprising at least one LCP layer may include, but are not limited to, substrates, adhesive layers, encapsulant layers, peelable layers, optoelectronic layers, photovoltaic layers, thermoplastic layers, and the like, and any combination thereof.

In some embodiments, suitable substrates for use in conjunction with layered articles of the present invention may include, but are not limited to, natural materials (e.g., wood), metals (e.g., copper, stainless steel, or aluminum), thermoplastic polymers (e.g., polyurethanes, low-density polyethylenes, linear low-density polyethylenes, ultra low-density polyethylenes, high-density polyethylenes, polypropylenes, polybutylenes, polyvinylidene fluoride polyethylene terephthalate, polystyrene, polyacrylamide, polymethyl methacrylate, polyacrylate, polymethacrylate, polyvinyl chloride, polyvinylidenechloride, polyacrylonitrile, polyvinyl alcohol, ethylene vinyl alcohol copolymer, nylon, or liquid crystal polymers), inorganic materials (e.g., glass or quartz), and the like, and any combination thereof.

In some embodiments, suitable adhesive layers for use in conjunction with layered articles of the present invention may include, but are not limited to, ethylene vinyl acetate copolymer adhesives, epoxy-type adhesives, urethane adhesives, pressure sensitive adhesives, glue, gelatin, caesin, starch, cellulose esters, aliphatic polyesters, poly(alkanoates), aliphatic-aromatic polyesters, sulfonated aliphatic-aromatic polyesters, polyamide esters, rosin/polycaprolactone triblock copolymers, rosin/poly(ethylene adipate) triblock copolymers, rosin/poly(ethylene succinate) triblock copolymers, poly(vinyl acetates), poly(ethylene-co-ethylacrylate), poly(ethylene-co-methylacrylate), poly(ethylene-co-propylene), poly(ethylene-co-1-butene), poly(ethylene-co-1-pentene), poly(styrene), acrylics, polyurethanes, sulfonated polyester urethane dispersions, nonsulfonated urethane dispersions, urethane-styrene polymer dispersions, non-ionic polyester urethane dispersions, acrylic dispersions, silanated anionic acrylate-styrene polymer dispersions, anionic acrylate-styrene dispersions, anionic acrylate-styrene-acrylonitrile dispersions, acrylate-acrylonitrile dispersions, vinylchloride-ethylene emulsions, vinylpyrrolidone/styrene copolymer emulsions, carboxylated and noncarboxylated vinyl acetate ethylene dispersions, vinyl acetate homopolymer dispersions, polyvinyl chloride emulsions, polyvinylidene fluoride dispersions, ethylene acrylic acid dispersions, polyamide dispersions, anionic carboxylated or noncarboxylated acrylonitrile-butadiene-styrene emulsions and acrylonitrile emulsions, resin dispersions derived from styrene, resin dispersions derived from aliphatic and/or aromatic hydrocarbons, styrene-maleic anhydrides, gamma-chloropropylmethoxysilane, vinyltrichlorosilane, vinyltriethoxysilane, vinyltris(beta-methoxyethoxy)silane, gamma-methacryloxypropyltrimethoxysilane, beta-(3,4-epoxycyclohexyl)ethyltrimethoxysilane, gammaglycidoxypropyltrimethoxysilane, vinyl-triacetoxysilane, gamma-mercaptopropyltrimethoxysilane, gamma-aminopropyltriethoxysilane, N-beta-(aminoethyl)-gamma-aminopropyl-trimethoxysilane, and the like, and any combination thereof. In some embodiments, ethylene vinyl acetate copolymer adhesives having a vinyl acetate content ranging from about greater than 0% to about 45%, including any subset therebetween, may be preferred.

In some embodiments, ethylene vinyl acetate copolymer adhesives may advantageously provide for LCP layers to be adhered to another layer with a lap strength of about 1 MPa or greater. In some embodiments, ethylene vinyl acetate copolymer adhesives may provide for LCP layers to be adhered to another layer with a lap strength ranging from a lower limit of about 1 MPa, 1.5 MPa, 2 MPa, or 3 MPa to an upper limit of about 10 MPa, 8 MPa, 5 MPa, 4 MPa, 3 MPa, 2.5 MPa, or 2 MPa, and wherein the lap strength may range from any lower limit to any upper limit and encompass any subset therebetween.

In some embodiments, suitable encapsulant layers for use in conjunction with the layered articles of the present invention may include, but are not limited to, ethylene vinyl acetate copolymer, polydimethylsiloxane, polyvinylbutyral, polyolefin elastomer, polyolefin elastomer, olefinic block copolymer, polyurethane, and the like, and any combination thereof.

In some embodiments, peelable layers for use in conjunction with the present invention may provide peelable seals for protecting adjacent materials. For example, the peelable layer could be a blend of two incompatible polymers, where the incompatibility of these polymers would sufficiently weaken this layer to allow it to fail cohesively when the film is pulled apart. One example of such a layer would be a blend of polybutene and ethylene vinyl acetate copolymer.

In some embodiments, suitable photovoltaic layers for use in conjunction with layered articles of the present invention may comprise photovoltaic materials that may include, but are not limited to, semiconductor photovoltaic materials, organic photovoltaic materials (single layer or multilayer), and the like, or any hybrid thereof. Said photovoltaic materials may be in flexible forms or structurally firm materials. Nonlimiting examples of semiconductor photovoltaic materials may include crystalline, polycrystalline, or amorphous forms or quantum dot assemblies of silicon, germanium, magnesium sulfide, zinc sulfide, cadmium sulfide, copper indium gallium sulfide, magnesium selenide, zinc selenide, cadmium selenide, copper indium gallium selenide, copper indium gallium diselenide, aluminum phosphide, gallium phosphide, indium phosphide, aluminium arsenide, gallium arsenide, indium arsenide, gallium antimonide, aluminum antimonide, indium antimonide, zinc telluride, cadmium telluride, and any combination thereof. Nonlimiting examples of organic photovoltaic materials may include highly conjugated molecules, highly conjugated polymers, phthalocyaninne derivatives, perylene derivatives, poly[2-methoxy-5-(2′-ethyl-hexyloxy)-p-phenylene vinylene], fullerenes (e.g., C₆₀ and higher including endofullerenes), elongated fullerenes (e.g., C₇₀ and higher including endofullerenes), carbon nanotubes (e.g., single-walled, double-walled, or multiwalled including endonanotubes like peapods), and the like, and any combination thereof.

In some embodiments, suitable thermoplastic layers for use in conjunction with layered articles of the present invention may comprise thermoplastic polymers that include, but are not limited to, polycarbonates, polyamides, polysulfones, polyphenylene sulfides, polyphenylene ethers, polyether ketones, polyetherimides, polycyclohexylene-dimethylene terephthalate, and the like, any derivative thereof, and any combination thereof.

By way of nonlimiting example, an LCP article according to at least some embodiments of the present invention may comprise, in order, an LCP layer described herein, an adhesive layer, and an encapsulant layer.

By way of another nonlimiting example, an LCP article according to at least some embodiments of the present invention may comprise, in order, a substrate, an adhesive, and an LCP article component described herein. For example, the LCP article component may, in some embodiments, be an LCP layer. In other embodiments, the LCP article component may be a molded LCP component, like a micro-channel mold.

By way of yet another nonlimiting example, an LCP article according to at least some embodiments of the present invention may comprise, in order, an LCP article component described herein, an adhesive layer, and a peelable layer.

In some embodiments, additional layers may be applied to an LCP article component by, for example, at least one of dipping, soaking, spraying, painting, washing, extruding, coextruding, extrusion coating, casting, laminating, compression molding, blow molding, adhering, and the like, any hybrid thereof, and any combination thereof.

By way of nonlimiting example an LCP article may, in some embodiments, be formed by laminating, in order, a first LCP layer, a first adhesive layer, a second LCP layer, a second adhesive layer, a third LCP layer, a third adhesive layer, and a peelable layer.

By way of another nonlimiting example, an LCP article may, in some embodiments, be formed by extruding an adhesive layer onto an LCP article component described herein.

By way of yet another nonlimiting example, an LCP article may, in some embodiments, be formed by extruding an adhesive layer onto an LCP layer described herein, and then laminating an additional layer described herein to the LCP layer/adhesive layer.

By way of another nonlimiting example, an LCP article may, in some embodiments, be formed by spraying an adhesive layer onto at least a portion of the surface of an LCP article component and then applying an additional layer to the adhesive layer. Further, in some embodiments, an LCP article may, in some embodiments, be formed by spraying an adhesive layer onto at least a portion of the surface of an additional layer and then applying an LCP article component to the adhesive layer.

In some embodiments, articles comprising an LCP composition of the present invention may be at least a portion of a product. Suitable products may include, but are not limited to, packaging (e.g., food packaging, beverage packaging, pharmaceutical/medical packaging (e.g., blister packs), chemical packaging, and electronics packaging), electronics or components thereof (e.g., circuit boards, flexible circuit boards, sockets, bobbins, switches, connectors, chip carriers, and sensors), bio-containers (e.g., blood bags, dialysis bags, saline bags, syringes, and vials), medical tubing (e.g., urinary catheters, cardiac catheters, intravenous catheters, feeding tubes, and dialysis tubing), surgical equipment, medical implants (e.g., joint replacements, RFID tags, pace makers, implantable sensors, and channeled drug delivery devices), electrophoretic devices, fuel cells, solar cells, safety garments, heat resistant belts, and the like, and any hybrid thereof. One skilled in the art, with the benefit of this disclosure, should understand that some products comprising LCP compositions may comprise layers and/or components not necessarily described herein.

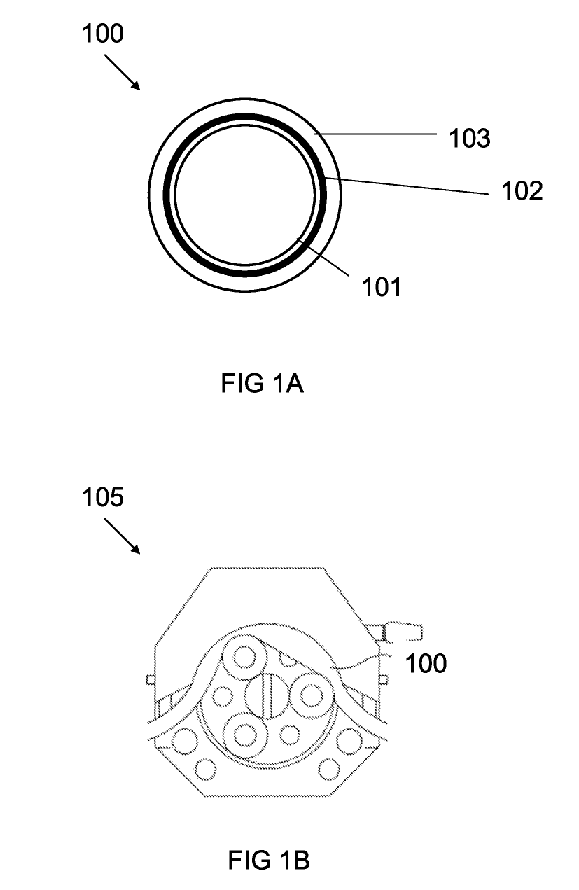

By way of nonlimiting example, as illustrated in FIG. 1A a medical tubing 100 (e.g., a urinary catheter, a cardiac catheter, a intravenous catheter, a feeding tube, tubes used in conjunction with biofluids (e.g., dialysis tubing), and the like) may, in some embodiments, comprise a tube 101 comprising LCP compositions described herein, an adhesive layer 102 disposed on at least a portion of the outer surface of the tube 101, and a thermoplastic layer 103 disposed on the adhesive layer 102. FIG. 1B illustrates the medical tubing 100 being used in conjunction with a peristaltic pump 105, e.g., as in dialysis or aphaeresis equipment. Further, in some embodiments, tubings like cardiac catheters and dialysis tubing may include the inner surface of the tubing having anticoagulant molecules disposed thereon. As used herein, the term “biofluid” refers to a biologically compatible fluid and/or a biological fluid, e.g., saline, a pharmaceutical agent in saline, whole blood, a component of whole blood, urine, and the like.

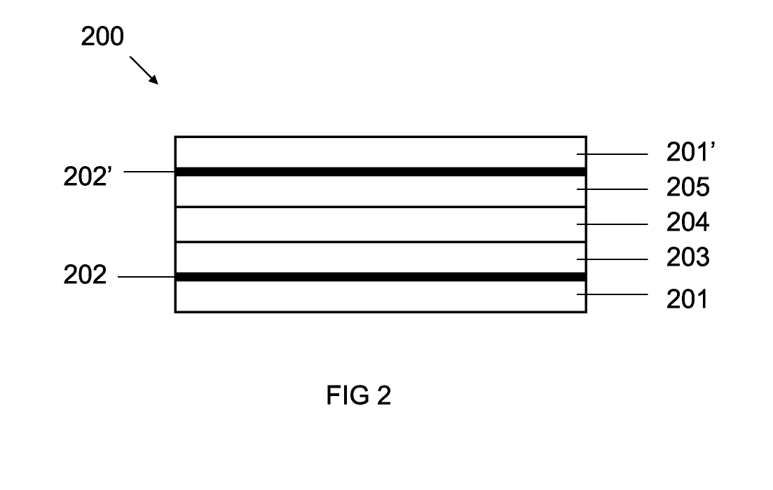

By way of another nonlimiting example, as illustrated in FIG. 2, an optoelectronic structure 200, in some embodiments, may comprise at least one LCP layer 201 described herein, an adhesive layer 202 disposed on at least a portion of a first LCP layer 201, and an optoelectronic device 203 disposed on the adhesive layer 202. Further, in some embodiments, the optoelectronic structure 200 may further comprise an encapsulant layer 204 disposed on the optoelectronic device 203, a second adhesive layer 202′ disposed on the encapsulant layer, and a second LCP layer 201′ disposed on the second adhesive layer 202′.

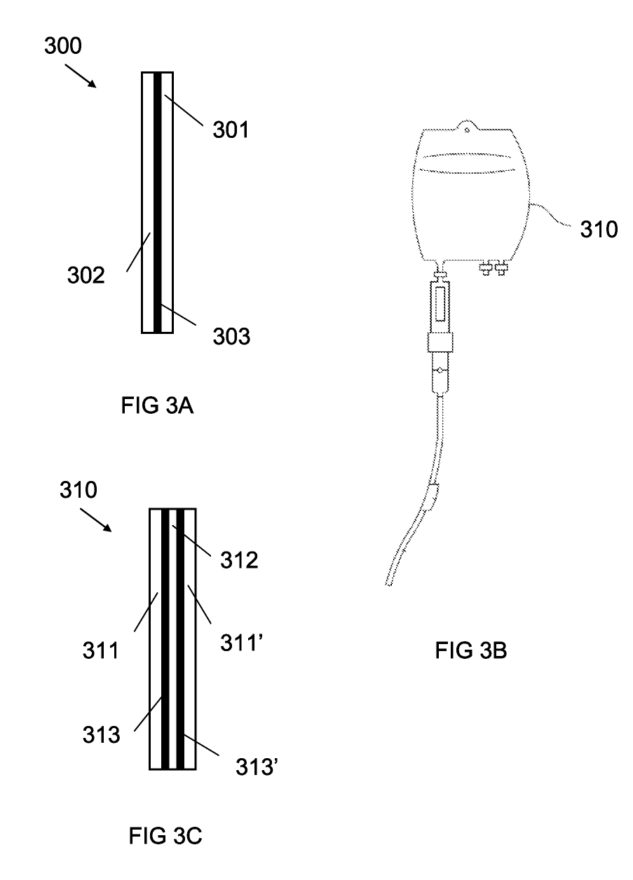

By way of yet another nonlimiting example, as illustrated in FIG. 3A, a bio-container 300 may, in some embodiments, comprise polymeric layer 301 and an LCP layer 302 described herein with an adhesive layer 303 disposed therebetween. In such a bio-container, the LCP layer may advantageously provide lower oxygen permeability and lower water vapor permeability than a bio-container without such an LCP layer. As used herein, the term “bio-container” refers to a container (e.g., a bag, a syringe, a vial, and the like) suitable for use in conjunction with holding, dispensing, and/or collecting biofluids. For example, a bio-container may be a bag 310 as illustrated in FIG. 3B-C (e.g., a saline bag, a saline/pharmaceutical agent bag, or a blood bag) that, in some embodiments, may comprise, in order, a first thermoplastic polymer layer 311, a first adhesive layer 313, an LCP layer described herein 312, a second adhesive layer 313′, and a second thermoplastic polymer layer 311′.

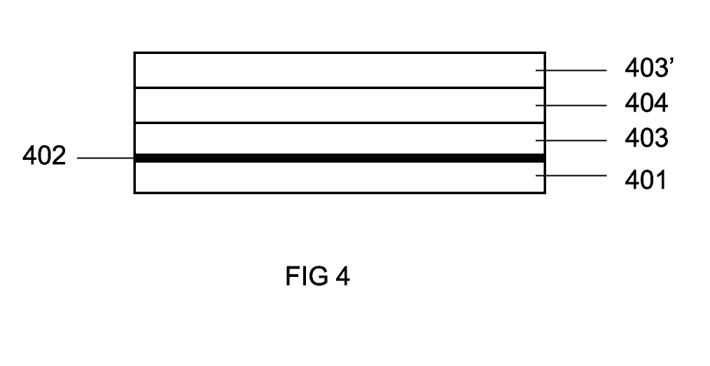

Referring now to FIG. 4, by way of another nonlimiting example, a solar cell may, in some embodiments, comprise, in order, an LCP layer 401 described herein, an adhesive layer 402 comprising a first ethylene vinyl acetate copolymer, and an encapsulant layer 403 comprising a second ethylene vinyl acetate copolymer. In some embodiments, a solar cell may further comprise a photovoltaic layer 404 and a second encapsulant layer 403′, wherein the photovoltaic layer 404 is disposed between the encapsulant layer 403 and the second encapsulant layer 403′.

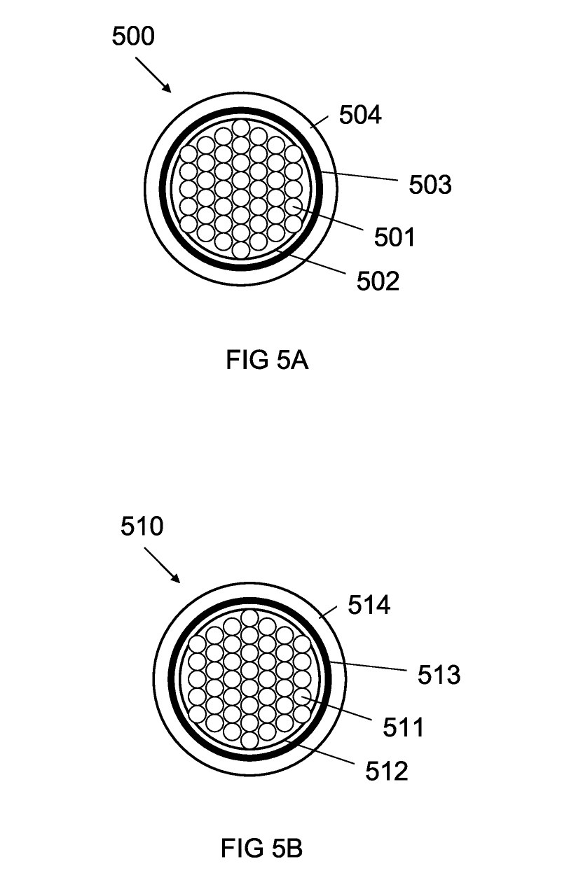

By way of yet another nonlimiting example, as illustrated in FIG. 5A, in some embodiments, a electrical wire 500 may, in some embodiments, comprise a plurality of conductive wires 501 having an insulating tube disposed there about, the insulating tube comprising an LCP tube 502 described herein, an adhesive layer 503 disposed on at least a portion of the outer surface of the LCP tube 502, and a thermoplastic layer 504 disposed on the adhesive layer 503. By way of another nonlimiting example, as illustrated in FIG. 5B, in some embodiments, a electrical wire 510 may, in some embodiments, comprise a plurality of conductive wires 511 having an insulating tube disposed there about, the insulating tube comprising an LCP tube 514 and an insulating fabric 512 adhered on at least a portion of the interior surface of the LCP tube 514 with an adhesive layer 513. In some embodiments, an electrical wire comprising an LCP article or component thereof may be used in conjunction with medical devices like pacemakers, neurostimulator, cochlear implants, and the like.

By way of another nonlimiting example, as illustrated in FIG. 6, a channeled structure 600 (e.g., an electrophoretic device, a micro-channeled medical device, a drug delivery device, a diagnostic device, and the like) may comprise a molded channel system 601 comprising LCP compositions described herein adhered to a substrate 602 with adhesive 603 so as to form at least one channel 604. In some embodiments, substrate 602 may comprise a second LCP composition described herein. In some embodiments, such a channeled structure may further comprise electrical components capable of applying electrical fields across the channel(s).

In some embodiments, products, articles, or components thereof that comprise at least one LCP layer described herein may be included in a kit that also includes a set of instructions. By way of nonlimiting example, medical tubing comprising PVC compounds of the present invention may, in some embodiments, be included in a kit with a set of instructions (e.g., how to install such tubing in a medical device like dialysis or aphaeresis equipment or how to properly use/insert a catheter). By way of another nonlimiting example, a bio-container comprising at least one LCP layer described herein in contact with an adhesive layer may, in some embodiments, be included in a kit with a set of instructions, perhaps including dosing information.

To facilitate a better understanding of the present invention, the following examples of preferred or representative embodiments are given. In no way should the following examples be read to limit, or to define, the scope of the present invention.

### EXAMPLES

### Example 1

A plurality of liquid crystal polymer compositions were prepared having a liquid crystal polymer and optionally 2,6-naphthalene dicarboxylic acid (“NDA”) in varying concentrations by weight of liquid crystal polymer. The liquid crystal polymers tested were (1) A950 (a liquid crystal polymer having no amide functionality, available from Celanese), (2) B950 (a liquid crystal polymer having amide functionality, available from Celanese), and (3) LKX1365 (a liquid crystal polymer having no amide functionality, available from Celanese). Table 1 provides physical properties of the liquid crystal polymer compositions.

TABLE 1

| | | Tensile | Flexure | Flexure |
|---|---|---|---|---|
| | Tensile Break | Modulus | Break Stress | Modulus |
| Sample | Stress (MPa) | (MPa) | (MPa) | (MPa) |
| A950 neat | 156.9 | 8750 | 162.5 | 10507 |
| A950 + 2% NDA | 171.9 | 8996 | 160.0 | 10355 |
| B950 neat | 169.0 | 18775 | 262.9 | 17645 |
| B950 + 2% NDA | 169.0 | 18738 | 248.2 | 17505 |
| LKX1365 neat | 150.0 | 12543 | 168.9 | 11963 |
| LKX1365 + 2% | 124.0 | 9773 | 134.5 | 10474 |
| NDA | | | | |

As shown in Table 1, the addition of NDA minimally affects the mechanical properties of the A950 and B950 liquid crystal polymers. However the addition of the late 2% NDA appears to strongly affect the mechanical properties of the LKX1365 liquid crystal polymer.

To test the lap strength, a tensile bar formed from each liquid crystal polymer composition was cut in half, and the smooth sides of each tensile bar (the gate area) where adhered to each other with an adhesive. The two tensile bar halves were clamped together using a binder clip and placed in an oven at 150° C. for 30 min to cure. The lap shear values were measured using ASTM Maximum Load Point methods with a pulling rate of 0.2 in/min at 23° C. Table 2 provides the maximum loading for each liquid crystal polymer/adhesive combination.

TABLE 2

| | Lap Strength (MPa) | | |
|---|---|---|---|
| LCP Sample\Adhesive | B-755 | 1838B/A | EVA |
| A950 neat | 1.02 | 1.23 | 0.97 |
| A950 + 2% NDA | 1.49 | 1.40 | 1.38 |
| A950 + 5% NDA | 1.19 | 1.12 | 0.77 |
| A950 + 10% NDA | 1.04 | 1.26 | 0.56 |
| A950 + 20% NDA | 0.83 | 1.33 | 1.02 |
| B950 neat | 0.99 | 1.12 | 1.53 |
| B950 + 2% NDA | 1.31 | 1.21 | 1.75 |
| B950 + 10% NDA | 0.88 | 1.49 | 1.55 |
| B950 + 20% NDA | 1.11 | 1.03 | 1.84 |

This example demonstrates that adhesion to a liquid crystal polymer composition can be increased with the addition of NDA. Further, this example illustrates that the concentration of NDA, or another adhesion enhancement compound, needed to increase the adhesion to liquid crystal polymer composition may be relatively low.

### Example 2

To test the lap strength of an liquid crystal polymer composition adhered to a metal using an ethylene vinyl acetate copolymer adhesive, a tensile bar was adhered to a piece of metal 1.45 mm thick having a bondline of 50 mm. Table 3 provides the lap strength of the various liquid crystal polymer/metal combinations.

TABLE 3

Lap Strength (MPa)

| LCP Sample\Metal | Aluminum | Copper |
|---|---|---|
| A950 | 0.46 | 0.92 |
| B950 | 0.44 | 1.01 |
| LKX1365 | 0.41 | 0.66 |

This example demonstrates that the addition of NDA liquid crystal polymers provide fair to good lap strength was adhered to a metal with an ethylene vinyl acetate copolymer adhesive.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. An article comprising:
a liquid crystal polymer article component that comprises a liquid crystal polymer composition that comprises:
a liquid crystal polymer; and
an adhesion enhancement compound; and
an adhesive layer disposed on at least a portion of the liquid crystal polymer article component.

2. The article of claim 1, wherein the liquid crystal polymer article component is in the form of at least one selected from the group consisting of a molded article, a tube, a fiber, a billet, a sheet, and any hybrid thereof.

3. The article of claim 1, wherein the adhesive comprises ethylene vinyl acetate copolymer.

4. The article of claim 1, wherein the liquid crystal polymer comprises at least one monomeric unit selected from the group consisting of a monomer according to Formula I, a monomer according to Formula II, a monomer according to Formula III, a monomer according to Formula IV, a monomer according to Formula V, a monomer according to Formula VI, a monomer according to Formula VII, and any combination thereof; 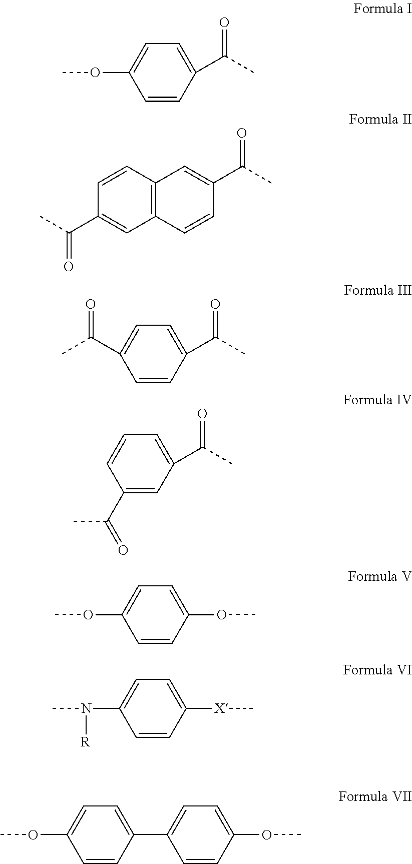
wherein X′ is selected from the group consisting of NR′, CO, O, and any combination thereof; and
wherein R and R′ are independently selected from the group consisting of H, alkyl groups having 1 to 4 carbon atoms, fluoroalkyl groups having 1 to 4 carbon atoms, phenyl, and any combination thereof.

5. The article of claim 1, wherein the liquid crystal polymer has an intrinsic viscosity ranging from about 0.1 dl/g to about 10 dl/g.

6. The article of claim 1, wherein the adhesion enhancement compound is at least one selected from the group consisting of, including any derivative thereof and any combination thereof, 2,6-naphthalene dicarboxylic acid, 6-hydroxyl naphthalene 2-carboxylic acid, p-hydroxybenzoic acid, terephthalic acid, isophthalic acid, benzenediol, a bisphenol, 4-aminophenol, acetaminophen, styrene, α-methyl styrene, halogenated styrene, acrylonitrile, methylacrylonitrile, acrylamide, methacrylamide, vinyl naphthalene, N-methylol acrylamide, vinyl acetate, vinylchloride, vinylidene chloride, benzyl acrylate, methacrylic acid, itaconic acid, fumaric acid, maleic acid, a compound according to Formula VIII, a compound according to Formula IX, a compound according to Formula X, a compound according to Formula X, a compound according to Formula XII, a compound according to Formula XIII, a compound according to Formula XIV, a compound according to Formula XV, and any combination thereof; 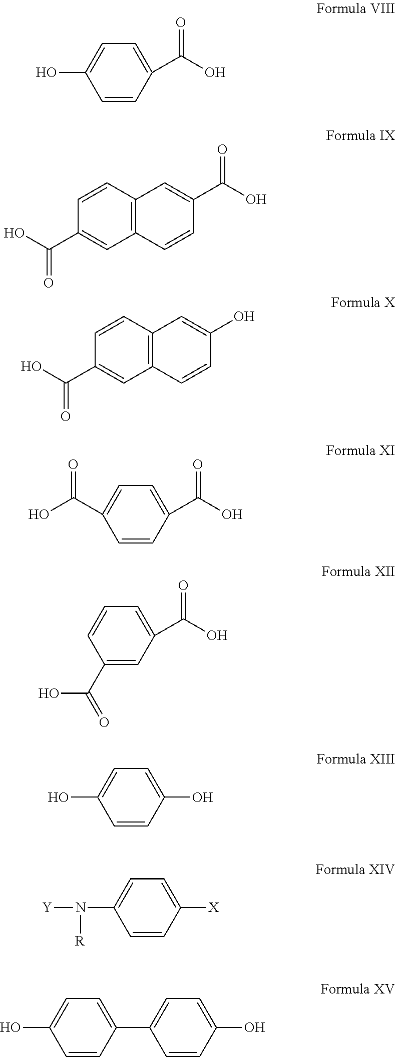
wherein X is selected from the group consisting of NY′R′, COOH, OH, and any combination thereof;
wherein Y and Y′ are independently selected from the group consisting of H, HOCH₃, and any combination thereof; and
wherein R and R′ are independently selected from the group consisting of H, alkyl groups having 1 to 4 carbon atoms, fluoroalkyl groups having 1 to 4 carbon atoms, phenyl, and any combination thereof.

7. The article of claim 1, wherein the adhesion enhancement compound is present in an amount ranging from about 0.01% to about 30% by weight of liquid crystal polymer.

8. The article of claim 1, wherein the liquid crystal polymer composition has a water vapor transmission rate of about 3 g/m²/day or less, as measured by ASTM R1249-06 with a 25 micron thick sample of the liquid crystal polymer composition.

9. The article of claim 1, wherein the liquid crystal polymer composition has an oxygen permeability of about 3 g/m²/day or less, as measured by ASTM D3985 with a 25 micron thick sample of the liquid crystal polymer composition.

10. The article of claim 1, wherein the liquid crystal polymer composition further comprising at least one selected from the group consisting of a reinforcing agent, a thermoplastic resin, a mold release-improving agent, a nucleating agent, an antioxidant, a stabilizer, a plasticizer, a slipping agent, a color protection agent, a coloring agent, an ultraviolet light absorber, an antistatic agent, a lubricant, a flame retardant, and any combination thereof.

11. The article of claim 1, wherein the liquid crystal polymer article component is a liquid crystal polymer layer with a thickness of about 10 microns to about 1500 microns.

12. The article of claim 1 further comprising:
a second layer such that the adhesive layer is disposed between the liquid crystal polymer article component and the second layer, the second layer comprising at least one selected from the group consisting of a substrate, an encapsulant layer, a peelable layer, an optoelectronic layer, a photovoltaic layer, a thermoplastic layer, and any combination thereof.

13. The layered article of claim 12, wherein the liquid crystal polymer article component is adhered to the second layer with a lap strength of about 1 MPa or greater.

14. A product comprising the article of claim 1.

15. The product of claim 14, wherein the product is at least one selected from the group consisting of packaging, food packaging, beverage packaging, medical packaging, blister packs, chemical packaging, electronics packaging, electronics or a component thereof, a circuit board, a flexible circuit board, a socket, a bobbin, a switch, a connector, a chip carrier, a sensor, a bio-container, a blood bag, a dialysis bag, a saline bag, a syringe, a vial, medical tubing, a urinary catheter, a cardiac catheter, an intravenous catheter, a feeding tube, a dialysis tubing, surgical equipment, a medical implant, a joint replacement, an RFID tag, a pace maker, an implantable sensor, a channeled device, a channeled drug delivery device, an electrophoretic device, a fuel cell, a solar cell, a safety garment, and heat resistant belts.

16. A layered article comprising, in order:
a liquid crystal polymer layer comprising a liquid crystal polymer and an adhesion enhancement compound;
an adhesive layer comprising a first ethylene vinyl acetate copolymer; and
an encapsulant layer comprising a second ethylene vinyl acetate copolymer.

17. The layered article of claim 16, wherein the high-temperature monomer is present in the liquid crystal polymer layer at a concentration of about 0.01% to about 30% by weight of the liquid crystal polymer layer.

18. The layered article of claim 16, wherein the liquid crystal polymer layer has water vapor permeability of less than about 1.0 g/m²*24 hr.

19. The layered article of claim 16, wherein the liquid crystal polymer layer has an oxygen permeability of about 3 g/m²/day or less, as measured by ASTM D3985 with a 25 micron thick sample of the liquid crystal polymer composition.

20. The layered article of claim 16, wherein the liquid crystal polymer layer has a thickness of about 1 microns to about 1500 microns.

21. The layered article of claim 16 further comprising:
a photovoltaic layer and a second encapsulant layer, wherein the photovoltaic layer is disposed between the encapsulant layer and the second encapsulant layer.

22. A solar cell comprising the layered article of claim 16.

23. A layered article comprising, in order:
a first liquid crystal polymer layer comprising a first liquid crystal polymer and a first adhesion enhancement compound;
a first adhesive layer comprising ethylene vinyl acetate copolymer;
a second liquid crystal polymer layer comprising a second liquid crystal polymer and a second adhesion enhancement compound;
a second adhesive layer comprising ethylene vinyl acetate copolymer;
a third liquid crystal polymer layer comprising a third liquid crystal polymer and a third adhesion enhancement compound; and
a third adhesive layer comprising ethylene vinyl acetate copolymer.

24. A method comprising:
applying an adhesive comprising to a liquid crystal polymer layer that comprises a liquid crystal polymer and an adhesion enhancement compound.

25. The method of claim 24, wherein the adhesive comprises ethylene vinyl acetate copolymer.

26. The method of claim 24, wherein the high-temperature monomer is present in the liquid crystal polymer layer at a concentration of about 0.01% to about 30% by weight of the liquid crystal polymer layer.

27. The method of claim 24, wherein the liquid crystal polymer layer has water vapor permeability of less than about 1.0 g/m²*24 hr.

28. The method of claim 24, wherein the liquid crystal polymer layer has an oxygen permeability of about 3 g/m²/day or less, as measured by ASTM D3985 with a 25 micron thick sample of the liquid crystal polymer composition.

29. The method of claim 24, wherein the liquid crystal polymer layer has a thickness of about 1 microns to about 1500 microns.

30. The method of claim 24 further comprising:
applying an optoelectronic to the adhesive.

31. The method of claim 24 further comprising:
applying a thermoplastic polymer to the adhesive.

32. The method of claim 24, wherein the liquid crystal polymer layer is at least one form selected from the group consisting of a molded article, a sheet, a tube, and a fiber.

33. The method of claim 24 further comprising:
producing a product comprising the liquid crystal polymer layer having the adhesive layer disposed thereon, wherein the product is one selected from the group consisting of packaging, food packaging, beverage packaging, medical packaging, a blister pack, chemical packaging, electronics packaging, an electronic component, a circuit board, a flexible circuit board, a bag for biological fluids, a blood bag, a dialysis bag, a saline bag, a medical device, a catheter, a piece of surgical equipment, a component of a piece of surgical equipment, a medical implant, a joint replacement, a fuel cell, a solar cell, a safety garment, a heat resistant belt, and any hybrid thereof.

34. A method comprising:
extruding an adhesive layer comprising ethylene vinyl acetate copolymer onto a liquid crystal polymer layer comprising a liquid crystal polymer and an adhesion enhancement compound.

35. The method of claim 34 further comprising:
laminating the liquid crystal polymer layer having adhesive disposed thereon with at least one layer selected from the group consisting of a substrate, an adhesive layer, an encapsulant layer, a peelable layer, an optoelectronic layer, a photovoltaic layer, a thermoplastic layer, a second liquid crystal polymer layer, and any combination thereof.

36. A method comprising:
laminating an adhesive layer between a liquid crystal polymer layer and a second layer, the adhesive layer comprising ethylene vinyl acetate copolymer, and the liquid crystal polymer layer comprising a liquid crystal polymer and an adhesion enhancement compound.

37. The method of claim 36, wherein a second layer comprises at least one selected from the group consisting of a substrate, an adhesive layer, an encapsulant layer, a peelable layer, an optoelectronic layer, a photovoltaic layer, a thermoplastic layer, a second liquid crystal polymer layer, and any combination thereof.

38. The method of claim 36, wherein the high-temperature monomer is present in the liquid crystal polymer layer at a concentration of about 0.01% to about 30% by weight of the liquid crystal polymer layer.

39. The method of claim 36, wherein the liquid crystal polymer layer has water vapor permeability of less than about 1.0 g/m²*24 hr.

40. The method of claim 36, wherein the liquid crystal polymer layer has an oxygen permeability of about 3 g/m²/day or less, as measured by ASTM D3985 with a 25 micron thick sample of the liquid crystal polymer composition.

41. The method of claim 36, wherein the liquid crystal polymer layer has a thickness of about 1 microns to about 1500 microns.

42. A method comprising:
arranging a plurality of layers such that at least two layers are an adhesive layer adjacent to a liquid crystal polymer layer, the adhesive layer comprising ethylene vinyl acetate copolymer, and the liquid crystal polymer layer comprising a liquid crystal polymer and an adhesion enhancement compound; and
laminating the plurality of layers together to form a layered article.

43. The method of claim 42, wherein a second layer comprises at least one selected from the group consisting of a substrate, an adhesive layer, an encapsulant layer, a peelable layer, an optoelectronic layer, a photovoltaic layer, a thermoplastic layer, a second liquid crystal polymer layer, and any combination thereof.

44. The method of claim 42, wherein the high-temperature monomer is present in the liquid crystal polymer layer at a concentration of about 0.01% to about 30% by weight of the liquid crystal polymer layer.

45. The method of claim 42, wherein the liquid crystal polymer layer has water vapor permeability of less than about 1.0 g/m²*24 hr.

46. The method of claim 42, wherein the liquid crystal polymer layer has an oxygen permeability of about 3 g/m²/day or less, as measured by ASTM D3985 with a 25 micron thick sample of the liquid crystal polymer composition.

47. The method of claim 42, wherein the liquid crystal polymer layer has a thickness of about 1 microns to about 1500 microns.

48. A catheter comprising, in order:
a liquid crystal polymer layer comprising a liquid crystal polymer and an adhesion enhancement compound;
an adhesive layer disposed about at least a portion of the liquid crystal polymer layer, the adhesive layer comprising ethylene vinyl acetate copolymer; and
a thermoplastic layer disposed about the adhesive layer.

49. The catheter of claim 48, wherein the thermoplastic layer is a polyurethane.

50. An optoelectronic structure comprising, in order:
a liquid crystal polymer layer comprising a liquid crystal polymer and an adhesion enhancement compound;
an adhesive layer disposed on at least a portion of the liquid crystal polymer layer, the adhesive layer comprising ethylene vinyl acetate copolymer; and
an optoelectronic device.

51. The optoelectronic structure of claim 50 further comprising:
an encapsulant layer disposed on the optoelectronic device;
a second adhesive layer disposed on the encapsulant layer; and
a second liquid crystal polymer disposed on the second adhesive layer, the second liquid crystal polymer layer comprising a second liquid crystal polymer and a second high-temperature monomer.

52. A blood bag comprising, in order:
a first thermoplastic polymer layer;
a first adhesive layer;
a liquid crystal polymer layer comprising a liquid crystal polymer and an adhesion enhancement compound;
an second adhesive layer disposed on at least a portion of the liquid crystal polymer layer; and
a first thermoplastic polymer layer.

53. A piece of surgical equipment comprising:
a tube comprising a liquid crystal polymer layer comprising a liquid crystal polymer and an adhesion enhancement compound.

54. A channeled structure comprising:
at least one channel formed by a molded liquid crystal polymer composition adhered to a substrate with an adhesive, the molded liquid crystal polymer composition comprising liquid crystal polymer and an adhesion enhancement compound.
